(19) United States
(12) Patent Application Publication
(10) Pub. No.: US 2009/0149880 A1
(43) Pub. Date: Jun. 11, 2009

(54) Occlusion System for Management of Rectal or Anal Incontinence

(75) Inventor: Fred Gobel, Wilhelmsfeld (DE)

Correspondence Address: Pauley Petersen & Erickson, 2800 Wesr Higgins Road, Sute 365, Hoffman Estates, IL, 60169, US

(73) Assignee: BC & D CONCEPTS GMBH, WIHELMSFELD (DE)

(21) Appl. No.: 12/226,236

(22) PCT Filed: Apr. 5, 2007
(86) PCT No.: PCT/EP2007/003099

§ 371 Date: Oct. 10, 2008

(30) Foreign Application Priority Data
Apr. 12, 2006 (DE) 20-2006005-951.2

(51) Int. Cl. A61M 29/00

(52) U.S. Cl. 606/197; 606/192

(57) ABSTRACT

An occlusion system for management of rectal or anal incontinence, including a shaft element with a distal end and a proximal end and with a lumen extending all the way from the distal end to the proximal end of the shaft element. A balloon is mounted adhesively and sealingly on the shaft element and can be filled with a filing medium via a filing line, wherein the balloon is formed from a tube section and is shaped so that when filled with the filing medium it forms a first balloon configuration as an intrarectal balloon and a second balloon configuration as an abutment balloon, which are delimited from each other by a constriction. The distal end of the shaft element is designed with an end cap with a tip and, at a distance from the tip, at least one gas inlet opening provides communication with the lumen of the shaft element. The proximal end of the shaft element is connected to an outlet tube which extends transverse to the longitudinal axis of the shaft element, and has outlet openings at its ends and communicates with the lumen.

### BACKGROUND OF THE INVENTION

1. Field of the Invention

This invention relates to an occlusion system for managing rectal, or respectively anal incontinence, including a shaft element with a distal end and a proximal end, a lumen extending continuously from the distal end to the proximal end of the shaft element, and a balloon fixedly and sealingly attached to the shaft element and that can be charged with a filler medium via a filler line. The balloon is of a hose section and when filled with the filler medium, it forms a first balloon deformation as an intra-rectal balloon and a second balloon deformation as a support balloon, which are delimited from each other by a constriction.

2. Discussion of Related Art

Up to now, rectal tampon devices made of a foam material or cellulose material capable of swelling and unfolding automatically are used for taking care of patients with rectal, or respectively anal incontinence, which are inserted by the patient in the form of a compressed, approximately suppository-shaped body through the anus into the rectum, and swell up partially by a multiple of their original volume, by absorbing intestinal secretions, so that sealing is assured. However, such swelled-up tampon bodies are very difficult and very painful for the patient to remove. Also, the tampon body, which is unfolded and swollen, is often considered to be irritating by the patient and triggers an impulse to defecate, or respectively triggers the defecation effect. Also, swollen tampon devices are not capable of being cleaned and are therefore one-way products, which cannot be used again, which leads to a large consumption of tampon devices of up to 10 per day.

Sealing elements using inflatable balloons are known. Thus, species-defining German Patent Reference DE 10 2004 033 425 A1 shows an occlusion system, in which a shaft element with a distal end and a proximal end, as well as lumens extending within the shaft element, are provided and which is introduced, with the distal end in front, into the rectum via the anus. At least one balloon is fixedly and sealingly arranged on the shaft element, which can be filled with a filler medium through a filler opening, wherein the balloon is made from an extruded hose section of plastic foil and is pre-formed so that, when filled with the filler medium, it forms a first balloon deformation as intra-rectal balloon, which provides sealing, and also forms a second balloon deformation as a support balloon, which is located outside of the anus. The two balloon deformations are delimited against each other by a constriction, wherein the deformation of the balloon with two balloon deformations and a constriction between them can be achieved by re-shaping the hose section in a so-called hollow body blowing process, or also by blow molding method.

However, manipulating the known occlusion system leaves room for improvement and furthermore, the removal of intestinal gases through the occlusion system is difficult, because it is necessary for the distal, as well as the proximal ends, to be designed to be open in order to assure the passage of intestinal gas through the shaft element of the occlusion system, which otherwise seals the intestines. Thus, fecal matter can easily enter into the shaft element, and then exits in an undesired manner via the proximal end, or else causes a stoppage of the gas passage within the continuous lumen of the shaft element.

### SUMMARY OF THE INVENTION

It is one object of this invention to further develop an occlusion system in accordance with a species so that, along with a simplified manipulation thereof, a dependable removal of intestinal gas from the rectum is possible, while easy cleaning of the occlusion system is simultaneously intended to assure repeated use for the patient.

For attaining this stated object, the design of an occlusion system for managing rectal, or respectively anal incontinence in accordance with this invention is proposed.

Advantageous embodiments and further developments of the occlusion system in accordance with this invention are discussed in this specification and in the claims.

In accordance with this invention, the distal end of the shaft element has an end cap with a tip, and at least one gas inlet opening which is spaced apart from the tip and communicates with the lumen of the shaft element. The undesired entry of fecal matter through the gas inlet openings into the continuous lumen is dependably counteracted by this end cap with gas inlet openings distanced from its tip, because with the spaced-apart arrangement of the gas inlet openings from the tip of the end cap, at least one gas inlet opening is located at an angle to the longitudinal axis L of the shaft element and the entry opening into the continuous lumen at the distal end, so that even with a column of stool resting on the end cap, the fecal matter cannot lead to a closure of the at least one gas inlet opening.

Furthermore, the proximal end of the shaft element is connected with an outlet tube, which extends transversely with respect to the longitudinal axis of the shaft element and has outlet openings at the end and in turn communicates with the lumen. The outlet tube, which preferably extends at a 90° angle with respect to the longitudinal axis of the shaft element, is used as a grip when inserting and removing the occlusion system of this invention. In the inserted state of the occlusion system the outlet tube comes to lie in the anal fold of the patient, where it is not felt as interfering. At the same time, a dependable removal of the intestinal gas entering the lumen of the shaft element through the at least one gas inlet opening is possible via the two exit openings at the end, wherein because of the angled-off course, possible contaminants which have entered into the shaft element are dependably prevented from exiting from the exit openings.

In accordance with one embodiment of this invention, the at least one gas inlet opening provided in the area of the end cap of the occlusion system in accordance with this invention is cut into the end cap transversely to the longitudinal axis of the shaft element.

However, in accordance with an alternative embodiment of the occlusion system in accordance with this invention, the end cap extends in a mushroom cap-shaped manner over the distal end of the shaft element, and the at least one gas inlet opening is formed in the area of the end cap which extends over the shaft element.

To assure the easy introduction of the occlusion system in accordance with this invention and to prevent injury, the tip of the end cap is preferably atraumatically designed.

To assure the multiple reuse capability of the occlusion system in accordance with this invention, easy cleaning is possible for a patient. For this purpose, the outlet tube has appropriate connectors at one end, but in particular at both its ends, for connecting a commercially available syringe, so that the syringe, filled with a cleaning fluid, can be connected to one end of the outlet tube, so that by actuating the syringe the entire gas-conducting system of the outlet tube as well as the shaft element with the end cap can be rinsed and thus effectively cleaned in the simplest manner.

For assuring good sealing of the occlusion system in accordance with this invention in the rectum, the ends of the hose section forming the balloon are sealingly fastened on the shaft element near the proximal end, for example glued or welded to the shaft element, so that the balloon extends along the shaft element in the direction toward the distal end, forming a constriction.

This fastening of the balloon, along with the formation of a constriction, known from German Patent Reference DE 10 2004 033 425 A1, has the result that the balloon, in this case the intra-rectal balloon, when filled with the filler medium, performs a rolling movement along the longitudinal axis of the shaft element, namely in the direction towards the proximal end thereof, which, with the intra-rectal balloon inserted into the rectum, is oriented in the direction toward the anus and thus represents a dependable seal of the rectum toward the outside. Reference to details of this rolling movement is made to German Patent Reference DE 10 2004 033 425 A1, the disclosure contents of which are here expressly included in this specification.

In accordance with an alternative design of the occlusion system in accordance with this invention, one end of the hose section forming the balloon is fastened on the shaft element near the distal end, and the other end of the hose section forming the balloon is fastened on the shaft element near the proximal end. Thus, it is preferred that the respective ends of the hose section forming the balloon are fastened on the shaft element while forming a pocket fold, which is preferably open toward the outside of the balloon. Based on this formation of a pocket fold at the distal and proximal ends of the hose section forming the balloon, the distal and proximal ends of the balloon, when charged with the filler medium, each perform an oppositely directed rolling movement along the longitudinal axis of the shaft element in the direction toward the constriction. Thus, in the inserted state of the occlusion system the intra-rectal balloon is placed sealingly on the inside of the anus and provides dependable sealing, while on the other side the support balloon is sealingly placed against the outside of the anus and provides solid seating of the occlusion element. This solid seating with dependable sealing is particularly enhanced if the sum of the axial rolling movements of the distal and proximal ends of the balloon, or respectively of the intra-rectal, or respectively support balloon, being formed there corresponds at least to the axial length of the constriction, or is greater than the latter. The axial length of the constriction is understood to be the longitudinal section of the constriction in the direction of the longitudinal axis of the shaft element.

Also, the proximal end of the balloon can be shaped so that, when charged with the filler medium, it is protectingly placed over the free end of the outlet tube, which increases the wearing comfort of the patient.

The balloon of the occlusion system in accordance with this invention is preferably made of a hose section of polyurethane of a wall thickness of 5 to 45 μm. It is also possible to employ multi-layered, welded-together foil layers, for example of polyethylene, polyvinyl chloride or polyurethane, for producing the hose section. Producing the hose section from silicon or natural latex by a dipping method is also possible.

In accordance with this invention, the end cap can be put on the distal end of the shaft element as a separate component and there can be suitably and permanently fixed in place, for example can be glued or welded.

In accordance with a further embodiment of this invention, the end cap can be shaped integrally on the distal end of the shaft element.

Also, if required, it is possible to provide an adsorbing material inside the continuous lumen for filtering the intestinal gas. Activated charcoal can be used for this purpose.

Also, the filler line can have a valve and a pilot balloon for detecting the filling pressure of the balloon, so that filling the balloon, as well as its emptying, can take place via a syringe connected to the filler line.

One advantage of the occlusion system in accordance with this invention is not only its dependable sealing caused by the unfolding of the two balloon shapes, but also an easy insertion, or respectively the easy pulling of the occlusion system out of the anus, provided that the filler medium had first been aspirated from the balloon by the syringe connected to the filler line.

To assure particularly comfortable wear for the patient, the shaft element has as narrow an exterior diameter as possible, for example an exterior diameter of approximately 4 to 8 mm, and the balloon shape forming the intra-rectal balloon has a diameter of approximately 30 to 80 mm and a length of up to 20 cm in the filled, but not extended state.

When making a shaft element which, to achieve comfortable wear is preferably thin, it can be designed with a thickening in the area of connection with the balloon in order to create a sufficient fastening surface for the balloon, or respectively for the ends of the hose section forming the balloon.

### BRIEF DESCRIPTION OF THE DRAWINGS

Further embodiments and details of the occlusion system in accordance with this invention are explained in view of the drawings, wherein:

FIG. 1 shows an occlusion system in a first embodiment of this invention, in a schematic representation;

FIG. 2 shows the end cap of the occlusion system in accordance with a second embodiment of this invention, in an enlarged representation; and

FIG. 3 shows a schematic representation a further embodiment of this invention.

### DETAILED DESCRIPTION OF THE INVENTION

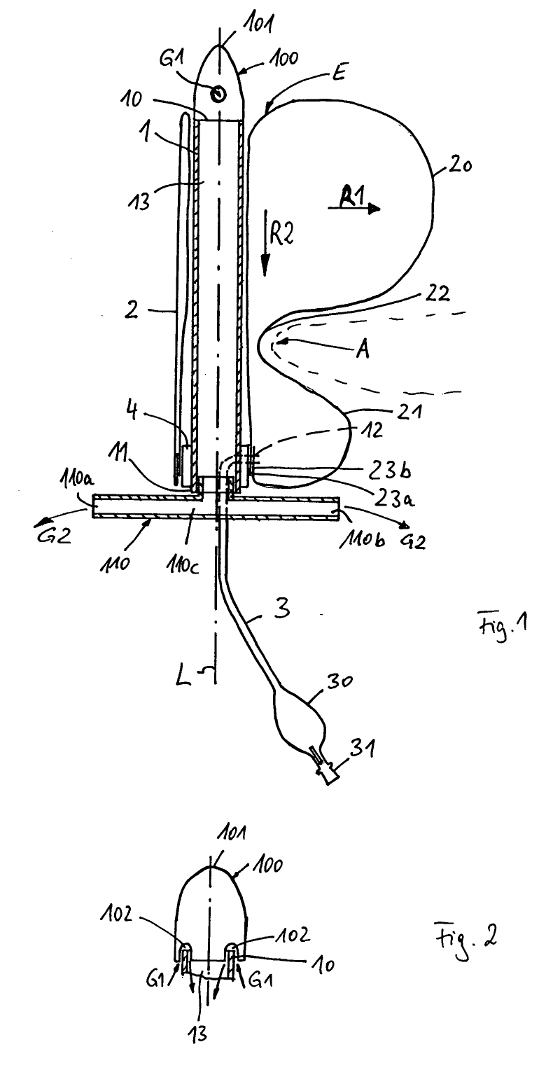

An occlusion system for managing rectal, or respectively anal, incontinence is represented in FIG. 1, and comprises a central shaft element 1, for example made as an elastically deformable plastic tube having a distal end 10 and a proximal end 11, as well as a lumen 13 extending continuously from the distal end 10 to the proximal end 11 and open at both ends.

A balloon 1, made from a hose section of a polyurethane foil of a wall thickness of, for example, 15 μm, is fastened in a manner to be described in greater detail later on the shaft element, and can be filled with a filler medium for expanding it via a filler line 3 and a corresponding perforation 12.

The distal end 10 of the shaft element 1 has an end cap 100, while an outlet tube 110, extending transversely with respect to the longitudinal axis L of the shaft element 1, is provided at the proximal end 11 of the shaft element 1.

The balloon 2 fastened to the shaft element 1 is shown in two states, which differ from each other, in the schematic sectional representation in accordance with FIG. 1, namely to the right of the center axis L in a expanded, such as charged with a filler medium, state, and to the left of the center axis L in its folded state, such as not charged with a filler medium.

To provide a sealing of the rectum in case of rectal, or respectively anal, incontinence, the occlusion system is inserted, with the end cap 100 fastened to the distal end 10 of the shaft element 1 at the front and with the balloon 2 present in the folded state, through the anus A into the rectum of the patient, wherein the occlusion system can be comfortably held by the patient by the outlet tube 110 provided in the area of the proximal end 11. In the end position, the outlet tube 110 comes to rest in the area of the anal fold of the patient, where it can be hardly felt.

In order to prevent injury when inserting the occlusion system and to cause the stretching of the anus, the end cap 100 has an atraumatic tip 101.

After inserting the occlusion system, a filler medium, for example air, can be introduced via the filler line 3 into the balloon 2, for which purpose an appropriate connector 31 with a valve is provided at the end of the filler line 3, so that a filled syringe containing air can be brought into contact here and air can be introduced by the syringe through the filler line 3 into the interior of the balloon 2. The filling pressure created inside the balloon 2 can be sensed by the patient with the aid of the pilot balloon 30 in the filler line 3.

The balloon 2 is made of a flexible hose section of a plastic foil, for example on the basis of polyurethane, wherein two hose ends 23a, 23b are together sealingly fastened on the shaft element 1 near the proximal end 11, and wherein the perforation 12 for introducing the filler medium runs into the interior of the balloon 2.

If, for increasing the wearing comfort, the shaft element 1 has a small exterior diameter of, for example, 4 to 8 mm, it is possible to provide a sufficient fastening surface for the ends 23a, 23b of the balloon to provide a thickened end piece 4 in this fastening area near the proximal end 11, on which the two ends 23a, 23b of the hose section are fastened, for example glued or welded to it.

With the above mentioned fastening of the proximal end 23a of the hose section, as well as the distal end 23b of the hose section, for shaping the balloon 2, the shaped balloon 2 is guided in the direction toward the distal end 10 of the shaft element while forming a constriction E resting against the shaft element 1, but is not fixedly guided by it outside of the thickened end piece 4, so that it is movable independently of the shaft element 1 to a large extent, and can be matched to the anatomy of the patient.

Also, the hose section forming the balloon 2 is pre-shaped into a shape during manufacture, for example by the so-called hollow body blowing process, or respectively the blow molding method, so that when filled with the filler medium 2 it forms balloon deformations 20, 21, which are separated from each other by a constriction 22, of which the balloon deformation 20 forms the intra-rectal balloon which, when the occlusion system is employed, unfolds inside the rectum of the patient, while the balloon deformation 21 comes to rest as a support balloon outside of the anus A of the patient and stabilizes the occlusion system.

With the described fastening of the balloon 2 on the shaft element 1 while forming the constriction E, when filled with a filler medium and correspondingly expanded in the arrow direction A, the balloon deformation 20 forming the intra-rectal balloon simultaneously performs a rolling movement directed along the longitudinal axis L of the shaft element in the direction of the proximal end 11 thereof in accordance with the arrow R, so that a dependable and greatly sealing contact with the anus A of the patient is caused, and the rectum is dependably closed from this direction.

For making it possible for this occlusion position of the occlusion system represented to the right of the center line L in FIG. 1 to divert rectal gases, the end cap 100 of the shaft element 1 is provided with at least one gas inlet opening 102, which communicates via the distal end of the shaft element with the continuous lumen 13 of the latter.

On the other hand, in the area of the proximal end 11 the continuous lumen 13 communicates with the continuous lumen 110c formed in the interior of the outlet tube 110, so that intestinal gas can enter in accordance with the arrow G1 into the continuous lumen 13 of the shaft element via the gas inlet opening 102 and can escape from there in accordance with the arrow G2 via the two outlet openings 110a, 110b of the outlet tube 110.

Preferably, at least one gas inlet opening 102 formed in the end cap 100 is arranged and spaced apart from the tip 101 of the end cap 100, in this case transversely with respect to the longitudinal axis L of the shaft element. With this spaced-apart arrangement, transversely with respect to the longitudinal axis L of the shaft element 1, the at least one gas inlet opening 102 cannot be blocked by contents of the intestine, even with a column of stool resting on the end cap 100, so that a permanent dependable gas removal is assured.

If in spite of this a transfer of intestinal secretions should occur via the gas inlet opening 102 into the lumen 13, it is dependably prevented from leaving through the outlet openings 110a, 110b because of the right-angled transition in the direction toward the proximal end 11.

If the above explained occlusion system is to be removed from the rectum, in a reverse manner the filler medium is aspirated out of the interior of the balloon 2 through the filler line 3, so that the folded state in accordance with the representation to the left of the longitudinal axis of the shaft element again occurs and the occlusion system can be pulled out of the anus A without large resistance. The entire occlusion system can subsequently be rinsed by applying a syringe filled with a cleaning fluid to one of the outlet openings 110a, 110b of the outlet tube 110. Thus both outlet openings 110a, 110b are designed in the form of commercially available connectors, so that the appropriate syringe connection can take place.

Based on this simple rinsing possibility and cleaning of the entire occlusion system, the latter is suitable for repeated use by the patient.

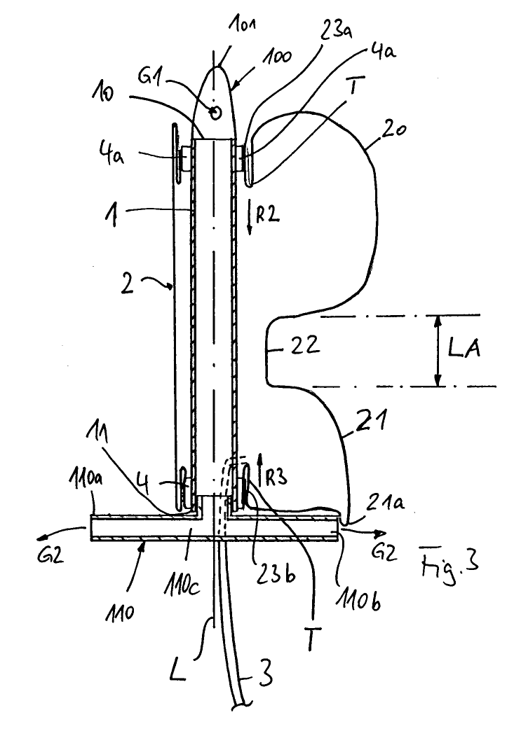

In an alternative embodiment of the end cap 100 of the occlusion system, represented in FIG. 2, the gas inlet openings 102 are not provided transversely with respect to the longitudinal axis L of the shaft element 1, the end cap 100 instead extends over the distal end 10 of the shaft element 1 in the manner of a mushroom cap, so that the end cap 100 has a certain radial projection, and the gas inlet openings 102 can then be formed in this projecting area of the end cap 100, so that it is possible for gases to move over in accordance with the arrows G1 into the continuous lumen 13 of the shaft element 1.

The occlusion of the gas inlet openings 102 by intestinal contents is also dependably prevented with this embodiment.

In each one of the above embodiments, the employed end cap 100 can be produced as a part separate of the shaft element 1 and can be fastened by fixed adhesion on the distal end 10 thereof, for example glued or welded on.

However, it is also possible to form such an end cap integrally on the distal end 10 of the shaft element 1.

A changed embodiment of the above explained occlusion system is represented in FIG. 3, in which like parts have like reference numerals and, for avoiding repetitions, are not again separately explained, unless this is necessary for understanding the embodiment.

The substantial difference between the embodiment in accordance with FIG. 1 and FIG. 3 is that the fastening of the hose section 2 forming the intra-rectal balloon 20 and the support balloon 21, which again can be formed as a polyurethane foil or a welded multi-layer foil, or also a silicon or natural latex material produced by a dipping method.

Fastening of the hose section 2 on the shaft element 1 takes place so that the distal end 23a of the hose section 2 adjoining the intra-rectal balloon 20 is fastened on the shaft element 1 near the distal end via thickenings 4a, while the proximal end 23b of the hose section 2 adjoining the support balloon 21 is fastened on the shaft element 1 near the proximal end 11 of the hose section 1 via thickenings 4.

Fastening of the distal, or respectively proximal ends 23a, b of the hose section 2 on the shaft element F1 takes place so that respectively one pocket fold T, which is open toward the outside of the hose section 2, is formed on the respective fastening point with the shaft element 1. The pocket fold T is responsible for the distal, or respectively proximal ends 23a, b of the hose section 2, or respectively the intra-rectal balloon 20 and the support balloon 21, being formed while filling with a filler medium, and perform an opposite rolling movement toward each other in accordance with the arrows R2, R3 in the direction toward the constriction 22, namely in an axial direction along the longitudinal axis L of the shaft element 1. These rolling movements in accordance with the arrows R2, R3 have as a result that, in the position of use of the occlusion system, the intra-rectal balloon 20 being formed rests sealingly from inside against the constriction 22, while the support balloon 21 rests against the anus from the outside in order for secure guidance of the occlusion element. Here, the size of the pocket folds T defines the possible rolling movement in the arrow direction R2, or respectively R3, and can be fixed by one skilled in the art in accordance with the needs. However, it is of particular advantage if the sum of the axial rolling movements R2, R3 is at least of the same size or greater than the axial length LA of the constriction 22 extending in the direction of the longitudinal axis L.

As shown in FIG. 3, the support balloon 21 has a formed-out portion 21a which, when opening the support balloon 21, is placed over the free end of the outlet tube 110 for increasing the wearing comfort for the patient and to receive the outlet tube in the manner of an air-filled cushion.

Besides the above explained functions of the occlusion system for managing rectal, or respectively anal incontinence, it is also suitable for the temporary management of hemorrhoidal bleeding, because with the balloon 2 it is possible to apply pressure which is pleasant for the patient and dependably prevents the entry of intestinal contents.

Furthermore, the above described occlusion system is also suitable for managing end-to-end anastomoses in case of near-anal colonic operations, in that the operation suture or the clips used are protected over several days against stool or secretions of the patient. Also, the removal of intestinal gases can occur during applications of radiotherapy, for example Seed radiation of the prostrate in combination with a rectally placed tissue displacement balloon.

1. An occlusion system for managing rectal or anal incontinence, comprising: a shaft element (1) with a distal end (10) and a proximal end (11), as well as a lumen (13) extending continuously from the distal end (10) to the proximal end (11) of the shaft element (1), a balloon (2) fixedly and sealingly attached to the shaft element (1) and chargeable with a filler medium via a filler line (3), the balloon (2) formed as a hose section and when filled with the filler medium forming a first balloon deformation (20) as an intra-rectal balloon and a second balloon deformation (21) as a support balloon delimited from each other by a constriction (22), the distal end (10) of the shaft element (1) including an end cap (100) with a tip (101), at least one gas inlet opening (102) spaced apart from the tip (101) and communicating with the lumen (13) of the shaft element (1), and the proximal end (11) of the shaft element (1) connected with an outlet tube (110) and extending transversely with respect to the longitudinal axis (L) of the shaft element (1) and having outlet openings (110a, b) at the end and communicating with the lumen (13).

2. The occlusion system in accordance with claim 1, wherein the at least one gas inlet opening (102) is cut into the end cap (100) transversely with respect to the longitudinal axis (L) of the shaft element (1).

3. The occlusion system in accordance with claim 1, wherein the end cap (1) extends in a mushroom cap-shaped manner over the distal (10) end of the shaft element (1), and the at least one gas inlet opening (102) is formed near the end cap (100) which extends over the shaft element (1).

4. The occlusion system in accordance with claim 3, wherein the tip (101) of the end cap (100) is atraumatically designed.

5. The occlusion system in accordance with claim 4, wherein the outlet tube (110) has connectors at both ends (110a, 110b) for connecting a syringe.

6. The occlusion system in accordance with claim 5, wherein the hose section forming the balloon (2) is of an at least single-layered plastic material.

7. The occlusion system in accordance with claim 6, wherein the balloon (2) is of a hose section of a polyurethane of a wall thickness of 5 μm to 45 μm.

8. The occlusion system in accordance with claim 7, wherein the ends (23a, 23b) of the hose section forming the balloon (2) are sealingly fastened on the shaft element (1) near the proximal end (11), so that the balloon (2) extends along the shaft element in a direction toward the distal end (10) while forming a constriction.

9. The occlusion system in accordance with claim 5, wherein the hose section forming the balloon (2) is of a silicon or a natural latex produced by a dipping method.

10. The occlusion system in accordance with claim 7, wherein one end (23a) of the hose section forming the balloon (2) is fastened near the distal end (10) on the shaft element (1), and the other end (23b) of the hose section forming the balloon (2) is fastened on the shaft element (1) near the proximal end (11).

11. The occlusion system in accordance with claim 10, wherein the ends (23a, 23b) of the hose section forming the balloon (2) are fastened on the shaft element (1) while forming a pocket fold (T) so that the distal end (10) and the proximal end (11) of the balloon (2) when charged with the filler medium perform a rolling movement (R2, R3) along the longitudinal axis (L) of the shaft element (1) toward the constriction (22), and a sum of the axial rolling movements (R2, R3) corresponds at least to an axial length (LA) of the constriction (22) or is greater than the axial length (LA).

12. The occlusion system in accordance with claim 11, wherein the end cap (100) is integrally shaped on the distal end (10) of the shaft element (1).

13. The occlusion system in accordance with claim 12, wherein an adsorbing material is provided inside the continuous lumen (3) of the shaft element (1).

14. The occlusion system in accordance with claim 13, wherein the filler line has a valve and a pilot balloon (30) for detecting a filling pressure of the balloon (2).

15. The occlusion system in accordance with claim 14, wherein the shaft element (1) has an exterior diameter of 4 mm to 8 mm, and the balloon deformation (20) forming the intra-rectal balloon has a diameter of approximately 30 mm to 80 mm and a length of 40 mm up to 20 cm in the filled, but not extended state.

16. The occlusion system in accordance with claim 15, wherein the shaft element (1) is designed with a thickening in the connecting area with the balloon (2).

17. The occlusion system in accordance with claim 1, wherein the end cap (1) extends in a mushroom cap-shaped manner over the distal (10) end of the shaft element (1), and the at least one gas inlet opening (102) is formed near the end cap (100) which extends over the shaft element (1).

18. The occlusion system in accordance with claim 1, wherein the tip (101) of the end cap (100) is atraumatically designed.

19. The occlusion system in accordance with claim 1, wherein the outlet tube (110) has connectors at both ends (110a, 110b) for connecting a syringe.

20. The occlusion system in accordance with claim 1, wherein the hose section forming the balloon (2) is of an at least single-layered plastic material.

21. The occlusion system in accordance with claim 1, wherein the balloon (2) is of a hose section of a polyurethane of a wall thickness of 5 μm to 45 μm.

22. The occlusion system in accordance with claim 1, wherein the ends (23a, 23b) of the hose section forming the balloon (2) are sealingly fastened on the shaft element (1) near the proximal end (11), so that the balloon (2) extends along the shaft element in a direction toward the distal end (10) while forming a constriction.

23. The occlusion system in accordance with claim 1, wherein the hose section forming the balloon (2) is of a silicon or a natural latex produced by a dipping method.

24. The occlusion system in accordance with claim 1, wherein one end (23a) of the hose section forming the balloon (2) is fastened near the distal end (10) on the shaft element (1), and the other end (23b) of the hose section forming the balloon (2) is fastened on the shaft element (1) near the proximal end (11).

25. The occlusion system in accordance with claim 24, wherein the ends (23a, 23b) of the hose section forming the balloon (2) are fastened on the shaft element (1) while forming a pocket fold (T) so that the distal end (10) and the proximal end (11) of the balloon (2) when charged with the filler medium perform a rolling movement (R2, R3) along the longitudinal axis (L) of the shaft element (1) toward the constriction (22), and a sum of the axial rolling movements (R2, R3) corresponds at least to an axial length (LA) of the constriction (22) or is greater than the axial length (LA).

26. The occlusion system in accordance with claim 1, wherein the end cap (100) is integrally shaped on the distal end (10) of the shaft element (1).

27. The occlusion system in accordance with claim 1, wherein an adsorbing material is provided inside the continuous lumen (3) of the shaft element (1).

28. The occlusion system in accordance with claim 1, wherein the filler line has a valve and a pilot balloon (30) for detecting a filling pressure of the balloon (2).

29. The occlusion system in accordance with claim 1, wherein the shaft element (1) has an exterior diameter of 4 mm to 8 mm, and the balloon deformation (20) forming the intra-rectal balloon has a diameter of approximately 30 mm to 80 mm and a length of 40 mm up to 20 cm in the filled, but not extended state.

30. The occlusion system in accordance with claim 1, wherein the shaft element (1) is designed with a thickening in the connecting area with the balloon (2).
